(19) United States
(12) Patent Application Publication
(10) Pub. No.: US 2024/0307153 A1
(43) Pub. Date: Sep. 19, 2024

(54) ORTHODONTIC SEPARATOR

(71) Applicant: CARERING TECHNOLOGIES LTD, Lod (IL)

(72) Inventors: Aharon ITZKOVITZ, Lod (IL); Ari KUPIETZKY, Jerusalem (IL)

(21) Appl. No.: 18/576,605

(22) PCT Filed: Jul. 12, 2022
(86) PCT No.: PCT/IL2022/050750

§ 371 Date: Jan. 4, 2024

(30) Foreign Application Priority Data
Jul. 22, 2021 (IL) 285087

(51) Int. Cl. A61C 7/10

(52) U.S. Cl. CPC A61C 7/10; A61C 2201/00

(57) ABSTRACT

An orthodontic separator including a substrate including at least one resilient material configured to produce and maintain an outward force for a predetermined time period between adjacent teeth, wherein the substrate includes a plurality of layers including polymer fibers having a fluoride compound incorporated therein or that coats a surface thereof. The plurality of layers is arranged concentrically and configured such that the plurality of layers of the substrate successively dissolve or disintegrate within the predetermined time period when disposed between those adjacent teeth, whereby the separator dissolves or disintegrates.

### FIELD OF THE INVENTION

The present invention relates to dental devices and more specifically to a spacer or separator device for spacing teeth.

### BACKGROUND OF THE INVENTION

Prior to putting orthodontic bands on teeth, it may be required to insert one or more teeth separators/spacers between certain teeth to shift the teeth so as to provide enough separation to facilitate putting the bands on certain teeth. The separator(s) are subsequently removed by the orthodontist within a two-week period.

Occasionally a patient will forget to return to the orthodontist and the separator/spacer will remain. The retained separator may move or shift to an undesirable location including lodging into the gingiva, which may cause gingivitis. If untreated, the condition can exacerbate to a more serious one including periodontitis and/or periodontal abscess and may even cause loss of a tooth.

There are mainly three types of separators: brass wire, orthodontic spring wire and elastomeric separators, the latter being the most commonly used. These separators are easily placed (inserted between adjacent teeth) and are available in a variety of sizes and shapes.

Examples of orthodontic separators are disclosed in U.S. Pat. Nos. 6,988,887 (Hansen, et al., 2006 Jan. 24); 8,029,081 (Ho, 2011 Oct. 4); 5,461,133 (Hammar, et al., 1995 Oct. 4); 8,388,341 (Keltgen, 2013 Mar. 5); 8,029,281 (Ho, 2011 Oct. 4); 3,758,947 (Kesling, 1973 Sep. 18): US 2001/049,081 (Krupp, 2001 Dec. 6); US 2003/157454 (Hansen, et al., 2003 Aug. 21); JP 2011183043 (Hiroe, et al, 2011 Sep. 22); 6,142,778 (Summer, 2000 Nov. 7); and US 2009/215002 (Mah, 2009 Aug. 27).

### SUMMARY OF THE INVENTION

The present invention relates to a dental/orthodontic separator for separating teeth that is dissolvable or disintegrates in the mouth.

The orthodontic separator is formed from a substrate (e.g. in the shape of a ring, a plate, a rolled or folded tape or film, or in the form of a sponge) that is configured to provide a separation force on adjacent teeth when inserted there-between and that dissolves or disintegrates in the oral cavity.

Because it is common for separators to be left between appropriate adjacent teeth for about a week or two in order to produce a suitable space there-between, it is preferable that the separator maintains an efficacious thickness in combination with suitable mechanical and/or material properties (e.g. resilience) for about a week, or longer, despite eventually dissolving or disintegrating, most preferably completely. In other words, the dissolution or disintegration of the separator when inserted should be slow enough to maintain an efficacious force on the adjacent teeth between which the separator is inserted for the above-mentioned period of time.

According to embodiments of one aspect of the invention, there is provided a dissolvable or disintegrable orthodontic separator. The separator includes at least one material forming a substrate configured to provide a force between teeth and configured such that at least some components of the substrate dissolve or disintegrate when disposed between those teeth whereby the substrate either dislodges from between the teeth or completely dissolves or disintegrates.

In some embodiments, the substrate includes, singularly or in any combination: polyvinyl alcohol (PVOH); polyacrylic acid (PAA) and derivatives thereof, including polyacrylamide (PAAm) and polymethacrylamide (PMAA); polyethylene oxide (PEO); silicone elastomers; crosslinked hyalauronic acid (X-HYA); and blends of polyglycolic acid, polylactic acid, and caprolactone.

In some embodiments, the substrate includes PVOH or derivatives thereof. In some embodiments, the PVOH is present at a concentration of about 50 wt % to about 95 wt % of the total weight of the substrate. In some embodiments, the PVOH or derivatives thereof is present at a concentration of about 50 wt % to 75 wt % of the total weight of the substrate. In some embodiments, the PVOH or derivatives thereof is present at a concentration of about 75 wt % to 90 wt % the total weight of the substrate.

In some embodiments, the substrate includes PAA or derivatives thereof.

In some embodiments, the PAA derivatives include PAAm and/or PMAA. In some embodiments, the substrate includes PEO or derivatives thereof. In some embodiments, the substrate includes a silicone elastomer. In some embodiments, the substrate includes crosslinked hyalauronic acid (X-HYA) or derivatives thereof.

In some embodiments, the substrate includes either one or a combination of polyglycolic acid; polylactic acid; and caprolactone or derivatives thereof.

In some embodiments, the substrate includes a cast film or a lamination prepared from a solution including water as a solvent.

In some embodiments, the substrate includes at least one resilience or flexibility enhancer. In some embodiments, the at least one enhancer includes a plasticizer or softening agent. In some embodiments, the plasticizer or softening agent includes any one or a combination of glycerol; glycol; xylitol; ethylene glycol; diethylene glycol; polyethylene glycol (PEG); polypropylene glycol (PPG); sorbitol; a poloxamer; and a polydiol or derivatives thereof.

In some embodiments, the substrate includes a dental care agent; and/or therapeutic agent; and/or active ingredient. In some embodiments, the dental care agent, therapeutic agent or active ingredient is any one or combination of a fluoride compound; chlorhexidine; lidocaine hydrochloride; triamcinolone; acetonide; miconazole nitrate; nystatin; eugenol oil; nicotine; and cannabidiol oil.

In some embodiments, the fluoride compound includes one or any combination of the following: sodium fluoride; sodium fluorophosphate; sodium monofluorophosphate; stannous fluoride; titanium tetrafluoride; silver diamine fluoride; and nano silver fluoride.

In some embodiments, the substrate includes a combination of 50-98 wt % PVOH; and one of the following or combination thereof: 2-10 wt % PEG; 25-40 wt % glycerol; and 10-40 wt % sorbitol.

In some embodiments, the substrate includes a coating. In some embodiments, the coating is a lubricating coating, which can facilitate insertion between teeth. In some embodiments, the lubricating coating includes fullerene-like tungsten disulfide nanoparticles and/or halloysite nanotubes.

In some embodiments, the substrate includes an anti-microbial coating. In some embodiments, the anti-microbial agent includes any one or a combination of an anti-bacterial agent; an anti-septic; fatty acid esters; and non-silver stearoyl lactylates in an effective antimicrobial amount.

In some embodiments, the substrate is configured to maintain a force, between the teeth in which the separator is inserted, for at least one week.

In some embodiments, the separator is formed in the shape of a ring, for example a toroidal-shaped ring or a ring with a circular cross-section; and in some embodiments, the separator is oval, square or rectangular.

According to embodiments of another aspect of the invention, there is provided a method of preparing a dissolvable or disintegrable orthodontic separator. The method includes combining about 50-85 wt % PVOH, about 1-20 wt % sodium fluoride and about 5-50 wt % PEG to form a substrate of the separator. Exemplary methods of forming the substrate include casting, laminating and extruding.

In some embodiments, one step in the method of the preparation of the cast film includes drying under vacuum. In some such embodiments, the drying is performed at an absolute pressure of about 10 mm Hg to 200 mm Hg. In some such embodiments, the drying is performed at an absolute pressure of about 20 mm Hg to 150 mm Hg. In some such embodiments, the drying is performed at an absolute pressure of about 30 mm Hg to 100 mm Hg. In some such embodiments, the drying is performed at an absolute pressure of about 30 mm Hg to 50 mmHg. In some such embodiments, the drying is performed at an absolute pressure of about 30 mm Hg.

In some embodiments, the substrate includes a cast film prepared from a solution including an organic solvent, such as dimethyl sulfoxide (DMSO), dimethylformamide (DMF), hexamethylphosphoramide (HMPT) or tetrahydrofuran (THF) and combinations thereof. In some such embodiments, the cast film is dried under conditions of low humidity.

In some such embodiments, sustained release refers to release of the active ingredient at a rate of about 0.1-1.0 mg/day.

In some embodiments, the separator is configured to provide a fluoride concentration in saliva in a range of about 0.02 to about 1.0 ppm.

In some embodiments, the separator is configured to release the fluoride compound over a period of at least one week. In some such embodiments, the spacer is configured to release the fluoride compound over a period of about 1-4 weeks, more preferably about 2-4 weeks.

In some embodiments, the separator is provided in the form of a ring or band. In some embodiments, the material of the separator is solid; in some embodiments, the material of the separator is a foam. In some embodiments, the material of the separator is configured to swell. In some such embodiments, the swelling is into a predetermined (desired) shape.

According to embodiments of another aspect of the invention there is provided a method of preparing an orthodontic separator. The method includes: preparing a substrate configured to provide a force between teeth and configured wherein at least some components of the substrate dissolves or disintegrates when disposed between those teeth whereby the substrate either dislodges from between the teeth or it completely dissolves or disintegrates.

In some embodiments, preparing the substrate includes, singularly or in any combination, using polyvinyl alcohol (PVOH); polyacrylic acid (PAA) and derivatives thereof, including polyacrylamide (PAAm) and polymethacrylamide (PMAA); polyethylene oxide (PEO); silicone elastomers; crosslinked hyalauronic acid (X-HYA); and blends of polyglycolic acid, polylactic acid, and caprolactone to form the substrate.

In some embodiments, preparing the substrate includes using PVOH or derivatives thereof.

In some embodiments, preparing the substrate includes using PVOH at a concentration of about 50 wt % to about 95 wt % of the total weight of the substrate. In some embodiments, preparing the substrate includes using PVOH at a concentration of about 50 wt % to 75 wt % of the total weight of the substrate. In some embodiments, preparing the substrate includes using PVOH at a concentration of about 75 wt % to 90 wt % the total weight of the substrate.

In some embodiments, preparing the substrate includes using PAA and derivatives thereof. In some embodiments, preparing the substrate includes using PEO or derivatives thereof. In some embodiments, preparing the substrate includes using a silicone elastomer. In some embodiments, preparing the substrate includes using crosslinked hyalauronic acid (X-HYA) or derivatives thereof.

In some embodiments, preparing the substrate includes using either one or a combination of polyglycolic acid; polylactic acid; and caprolactone or derivatives thereof. In some embodiments, preparing the substrate includes using either one or a combination of PAAm and PMAA or derivatives thereof.

In some embodiments, preparing the substrate includes casting or laminating.

In some embodiments, preparing the substrate includes adding at least one resilience or flexibility enhancer.

In some embodiments, preparing the substrate includes adding at least one plasticizer or softening agent. In some embodiments, adding the plasticizer or softening agent includes adding any one or a combination of glycerol; glycol; xylitol; ethylene glycol; diethylene glycol; polyethylene glycol (PEG); polypropylene glycol (PPG); sorbitol; a poloxamer; and a polydiol or derivatives thereof.

In some embodiments, preparing the substrate includes adding a dental care agent; and/or therapeutic agent; and/or active ingredient. In some embodiments, adding a dental care agent; and/or therapeutic agent; and/or active ingredient includes adding any one or combination of a fluoride compound; chlorhexidine; lidocaine hydrochloride; triamcinolone; acetonide; miconazole nitrate; nystatin; eugenol oil; nicotine; and cannabidiol oil. In some embodiments, adding the fluoride compound includes adding one or any combination of the following: sodium fluoride; sodium fluorophosphate; sodium monofluorophosphate; stannous fluoride; titanium tetrafluoride; silver diamine fluoride; and nano silver fluoride.

In some embodiments, preparing the substrate includes adding a combination of 50-98 wt % PVOH and one of the following or combination thereof: 2-10 wt % PEG; 25-40 wt % glycerol; and 10-40 wt % sorbitol.

In some embodiments, preparing the substrate includes applying a coating. In some embodiments, applying the coating includes applying a lubricating coating.

In some embodiments, applying the lubricating coating includes applying fullerene-like tungsten disulfide nanoparticles and/or halloysite nanotubes.

In some embodiments, preparing the substrate includes applying an anti-microbial coating. In some embodiments, applying the anti-microbial coating includes applying any one or a combination of an anti-bacterial coating; an anti-septic; fatty acid esters; and non-silver stearoyl lactylates in an effective antimicrobial amount.

In some embodiments, wherein preparing the substrate includes compounding and extruding.

In some embodiments the method includes: dissolving PVOH in water to form an aqueous PVOH solution; adding an active ingredient to the PVOH solution to form a solution including PVOH and the active ingredient; forming a film or substrate from the solution including PVOH and the active ingredient; and drying the film to produce a substrate from which the separator is formed/shaped.

In some such embodiments, instead of an aqueous solution, the dissolving of PVOH is in an organic solvent. In some such embodiments, drying is carried out by slow evaporation. In some such embodiments, the organic solvent is dimethyl sulfoxide (DMSO); dimethylformamide (DMF); hexamethylphosphoramide (HMPT); tetrahydrofuran (THF); or any combinations thereof.

The terms “sustained release”; “extended release”; and “slow release”, and their derivatives, refer to a release of the active ingredient at a rate of about 0.02 to about 1 mg/day; and alternatively, refers to delivery of an active ingredient or active agent, at a rate sufficient to maintain a pharmaceutically-effective concentration of the active ingredient over a specific time period. In a preferred embodiment, the specific time period is at least one and more specifically about 1-4 weeks.

As used herein, the terms “treating”, “treatment”, derivatives thereof and similar terms, include preventing, curing, ameliorating, mitigating, or reducing the instances, rate or severity of a dental symptom or condition; or improving such dental symptom or condition, including for example, reducing the incidence or severity of dental caries.

As used herein, the term “remineralization” refers to deposition of minerals into previously damaged areas of a tooth. As used herein, the term “demineralization” refers to loss of minerals from an area of a tooth.

As used herein, when a numerical value or range is preceded by the term “about”, it is intended to indicate +/−10% of that value.

### BRIEF DESCRIPTION OF THE DRAWINGS

Some embodiments of the invention are described herein with reference to the accompanying figures. The figures are for the purpose of illustrative discussion and no attempt is made to show structural details in more detail than is necessary for a fundamental understanding of the invention. For the sake of clarity, some objects depicted in the figures are not necessarily drawn to scale.

FIG. 1 is a simplified schematic illustration of a dissolvable or disintegrable orthodontic separator in accordance with the principles of the present invention.

FIG. 2 is a cross-sectional illustration of the separator of FIG. 1.

FIG. 3 is a simplified schematic illustration of an alternative embodiment of the separator in accordance with the principles of the present invention.

FIG. 4 is a simplified schematic illustration of a further alternative embodiment of the separator in accordance with the principles of the present invention.

FIG. 5 is a perspective view of the separator, in the form of a toroidal ring, in accordance with the principles of the present invention.

FIG. 6 is a perspective view of the separator, in the form of a toroidal ring with wings, in accordance with the principles of the present invention.

FIG. 7 is a perspective view of the separator, having a washer-shape, in accordance with the principles of the present invention.

FIG. 8 is a perspective view of an exemplary separator of the present invention being inserted between teeth.

The principles, uses and implementations of the teachings herein may be better understood with reference to the accompanying description and figures. Upon perusal of the description and figures present herein, one skilled in the art should be able to implement the invention without undue effort or experimentation. In the figures, like reference numerals refer to like parts throughout.

### DETAILED DESCRIPTION

It is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

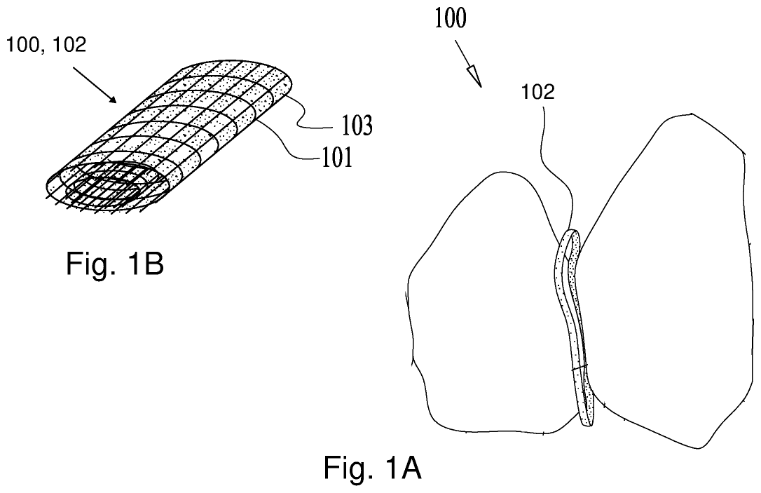

FIGS. 1-7 show embodiments of a dissolvable or disintegrable orthodontic separator (also referred to as a dental spacer) of the present invention. The separator is configured to provide a separation force between adjacent teeth when inserted between those teeth, while at the same time being configured to dissolve or disintegrate in the mouth. Separators are used to provide a space between teeth to allow for orthodontic bands to be conveniently applied. As such, the separators cannot dissolve or disintegrate at too rapid a rate in order that they will have some combination of thickness and material properties (or mechanical design) adequate to provide the above-mentioned separation force.

It is noted that a preferable separator will be configured to be conveniently insertable between closely adjacent teeth, as the role is to separate closely adjacent teeth. In some embodiments, for such purpose, the separator includes a smooth or low-friction surface coating and/or is adequately compressible. In addition, the separator has a configuration, which may include inherent material properties, to produce an outward force to press the adjacent teeth apart, for example a mechanical design and/or a suitable resiliency or elasticity.

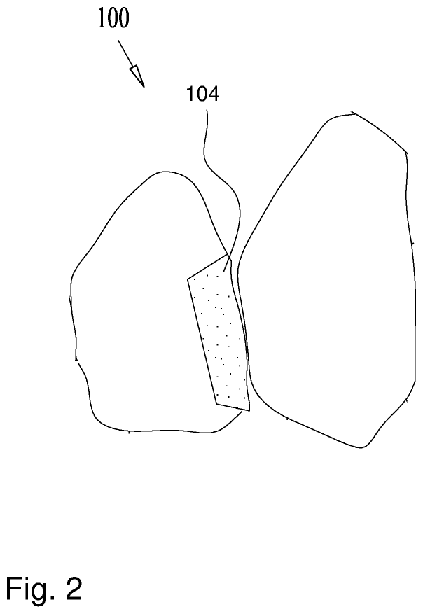

FIGS. 1 and 2 show a separator 100 embodied by a tape or strip in a rolled or folded configuration. Although it is envisioned that a preferably designed separator 100 will also include a material that has resilient properties, the design of a rolled or folded strip/tape itself can provide an outward force.

In addition, or alternatively, separator 100 includes a resilient polymeric material. Suitable polymeric materials include, singularly or in combination, including derivatives thereof: polyvinyl alcohol (PVOH); polyacrylic acid (PAA), e.g. polyacrylamide (PAAm); polymethacrylamide (PMAA); polyethylene oxide (PEO); silicone elastomers (e.g. DOWASIL™); crosslinked hyalauronic acid (X-HYA); and blends of polyglycolic acid, polylactic acid, and caprolactone (i.e. polycaprolactone or blends with lactide). In some embodiments, the separator further includes at least one plasticizer or softening agent, and/or other additives. In some embodiments, some or all of the above materials at least partially dissolve or disintegrate when disposed between the teeth, whereby the separator dislodges from between the teeth. In this regard, any non-dissolving/disintegrating components included in the separator may dislodge from between the teeth so as not to cause a dental pathology.

In some embodiments, separator 100 includes one or more resilience or flexibility enhancers, such as plasticizers and/or softeners. Exemplary plasticizers/softeners include glycols and glycerols, for example ethylene glycol; diethylene glycol; polyethylene glycol (PEG).

In some embodiments, separator 100 includes one or more active ingredients or dental agents, for example: sodium fluoride; sodium fluorophosphate; and stannous fluoride (including any combination thereof).

In some embodiments, separator 100 includes a combination of about 50-85 wt % PVOH, about 1.0-20 wt % sodium fluoride and about 5-50 wt % PEG or glycerol or sorbitol (or combinations thereof) to form a substrate of the separator.

Separator 100 may be manufactured by a variety of conventional polymer processing techniques, including, for example, extrusion; injection molding; liquid injection molding; compression molding; reaction injection molding; pressing; drawing; and die cutting.

Coatings may include antimicrobial coatings (e.g. an anti-bacterial coating; an anti-septic; fatty acid esters such as sodium stearoyl lactylate and/or other non-silver stearoyl lactylates in an effective antimicrobial amount are described herein).

In some embodiments, the anti-microbial coating includes an effective amount of a selenium compound. In some embodiments, the selenium compound is either or a combination of 3-[3-(2-{1-Methyl-2-[2-(2-methyl-acryloyloxy)-ethoxycarbonyl]-ethoxycarbonyl}-ethyldiselenyl)propionyl oxy]-butyric acid 2-(2-methyl-acryloyloxy)-ethyl ester (diSeAAEMA) and Di(cyclohexylmethyl-selenide). In some embodiments, the selenium compound is configured to allow for the formation of a selenium anion and/or free radical species, which may permanently attachment of the selenium anion to the surface of the orthodontic separator.

In some embodiments, the coating is a lubricating coating. The lubricating coating includes fullerene-like tungsten disulfide nanoparticles and/or halloysite nanotubes.

In some embodiments, the coating is combination of an anti-microbial coating and a lubricating coating.

FIG. 1A shows a dental device 100 (also referred to as a dental insert), in accordance with the embodiments of the present invention. Dental device 100 is configured to provide an extended release of an active ingredient in a sufficient dose (release rate) over an extended period. Dental device 100 includes a substrate 101 (e.g. made of polyvinyl alcohol-PVOH) and may further include an active compound or ingredient 103 incorporated therein. In some embodiments, active ingredient 103 is any one or combination of a fluoride compound; chlorhexidine; lidocaine hydrochloride; triamcinolone; acetonide; miconazole nitrate; nystatin; eugenol oil; nicotine; and cannabidiol oil.

In a preferred embodiment, active ingredient 103 is a fluoride compound.

Orthodontic separator 100 may be provided in any suitable shape, such as a square, rectangle, ring or the like, including formed in the shape of a ring, for example a toroidal-shaped ring or a ring with a circular cross-section.

In some embodiments, orthodontic separator 100 is in the form of a rolled film 102 (also interchangeably referred to herein as a band or ring), as illustrated in FIG. 1A. Band 102 may be soaked in alcohol (for example ethanol) to soften the band prior to insertion between the teeth. Band 102 may additionally or alternatively be coated with a suitable lubricant, which can make installation between teeth easier.

In a preferred embodiment, orthodontic separator 100 is configured to be inserted between adjacent teeth. In some embodiments, band 102 has a diameter in the range of about 3-6 mm and a thickness in the range of about 0.75-2.0 mm. In some embodiments, band 102 has a diameter of about 3 mm and a thickness of about 0.75 mm. In some embodiments, band 102 has a diameter of about 4 mm and a thickness of about 1 mm. In some embodiments, band 102 has a diameter of about 6 mm and a thickness of about 2 mm.

In some embodiments, substrate 101 is prepared from a solution including PVOH (polyvinyl alcohol) at a concentration of about 2-10 wt % of the total weight of the solution. In some embodiments, the PVOH is present at a concentration of about 10 wt % of the total weight of the solution. In some embodiments, following drying of the solution to form (PVOH) substrate 101, the PVOH is present at a concentration of about 50 wt % to about 99 wt % of the total weight of the PVOH substrate.

Orthodontic separator 100 may be configured to provide sustained release of active ingredient 103 when orthodontic separator 100 is retained within the oral cavity of a subject. The release rate of active ingredient 103 may be any rate sufficient to maintain an active ingredient concentration in the oral cavity having a beneficial effect such as for treating and/or preventing dental caries. In some embodiments, wherein active ingredient 103 is a fluoride compound, the total fluoride content in dental separator 100 is about 1.5-5.0 mg, for example 3.5 mg. In some such embodiments, the release rate is in the range of about 0.02 to about 1 mg/day fluoride. In some embodiments, Substrate 101 is configured to provide the fluoride compound to the oral cavity of a subject at a rate in a range of about 0.02-1.0 mg per day.

In some embodiments, active ingredient 103 includes a fluoride compound selected from the group consisting of sodium fluoride; sodium fluorophosphates; stannous fluoride; titanium tetrafluoride; silver diamine fluoride; nano silver fluoride; and combinations thereof. In some embodiments, active ingredient 103 includes sodium fluoride.

In some embodiments, the concentration of active ingredient 103 in substrate 101 is any concentration which is sufficient to achieve remineralization of the enamel of the teeth. In some embodiments, wherein active ingredient 103 is a fluoride compound, the concentration of the fluoride compound in substrate 101 is selected to provide a fluoride concentration in saliva in a range of about 0.02-1.0 ppm continuously for a period of at least about 1-4 weeks.

In some embodiments, wherein active ingredient 103 includes a fluoride compound, Substrate 101 is prepared from a solution including a fluoride compound at a concentration of about 0.1-20 wt % of the total weight of the solution.

In some embodiments, following drying of the solution to produce substrate 101, the fluoride compound is present at a concentration of about 0.5 wt % to about 20 wt % of the total weight of the substrate.

In some embodiments, active ingredient 103 is present in substrate 101 at a concentration in the range of about 100 to about 1000 ppm, including about 200 to about 600 ppm.

In some embodiments, the concentration of active ingredient 103 is selected such that the amount of active compound 103 released is sufficient only to act locally at a selected location.

In some embodiments, substrate 101 is configured to release active ingredient 103 in an amount of about 0.01-1.0 mg/day over a period of about 1 to about 4 weeks, such as about 2 to about 3 weeks.

In some embodiments, substrate 101 further includes a plasticizer. In some such embodiments, the plasticizer is selected from the group consisting of ethylene glycol, diethylene glycol, glycerol, polyethylene glycol (PEG), polypropylene glycol (PPG), Pluronic™ or polydiol materials and combinations thereof. In some embodiments, substrate 101 is prepared from a solution wherein the plasticizer includes polyethylene glycol (PEG), such as PEG 400, PEG 600 or PEG 800. In some embodiments, the plasticizer is present at a concentration of about 0.5 wt % to about 2 wt % of the total weight of the solution. In some embodiments, the plasticizer includes PEG 400. In some embodiments, the PEG is present at a concentration of about 2 wt % of the total weight of the solution.

In some embodiments, following drying of the solution to form substrate 101, the plasticizer, such as PEG, is present at a concentration of about 8 wt % to about 20 wt % of the total weight of the substrate.

In some embodiments, orthodontic separator 100 includes PVOH substrate 101 in the form of a film or a ring, cast or extruded, having active ingredient 103, such as a fluoride compound, incorporated therein. In some such embodiments, the film is a cast film prepared from a solution including PVOH and a fluoride compound and optionally a plasticizer dissolved in a suitable solvent. In some embodiments, the solvent is an aqueous solvent.

In some embodiments, the cast film is dried at about 50° C. under vacuum, following casting.

In some embodiments, the film is prepared by melt processing or thermal pressing (also referred to as heat pressing). In such embodiments, molten PVOH is mixed with the active ingredient and optionally a plasticizer in the proportions that are desired for preparation of substrate 101.

In some embodiments, substrate 101 is stable over a broad pH range of about 2-10, including about 4-9. Substrate 101 is considered to be stable when a change in dissolution rate of the substrate in saliva is no more than 5% greater than the dissolution rate of the substrate in water at pH 7.

In some embodiments, orthodontic separator 100 includes a substrate 101 including a cast film prepared from a PVOH solution, wherein the cast film is first dried and the active ingredient 103 is incorporated into the film after drying, such as by absorption from an aqueous NaF solution.

In some embodiments, substrate 101 is a material including a non-woven fabric made of polymer fibers having fluoride compound 103 incorporated therein.

In some such embodiments, fluoride compound 103 is embedded within the polymer i.e. dispersed within a solid polymeric matrix.

In some embodiments, substrate 101 includes a plurality of distinct polymer layers. In some such embodiments, substrate 101 is provided as a plurality of concentrically arranged layers, such as shown in FIG. 1B. In some such embodiments, fluoride compound 103 is incorporated into at least two, or into each of the plurality of layers, such as by absorption.

In some embodiments, substrate 101 may include micro-channels within the polymer. In some such embodiments, fluoride compound 103 may be released from such channels.

In some embodiments, fluoride compound 103 coats the surface of the polymer fibers.

In some embodiments wherein substrate 101 includes a plurality of layers, the outer layer of substrate 101 initially dissolves, disintegrates or hydrolyzes upon contact with saliva, thereby releasing fluoride compound 103 therefrom exposing an underlying layer to contact with the saliva. A first underlying layer is then dissolved or disintegrates to release fluoride compound 103 contained therein to expose a subsequent underlying layer, and so forth, until all layers of separator 100 dissolve or disintegrate.

In some embodiments, substrate 101 includes a plurality of polymer fibers, wherein fluoride compound 103 is incorporated within or coats the surface on each fiber. The fibers may be concentrically arranged, such that an outer peripheral layer dissolution or disintegration first, thereby releasing fluoride compound 103 and exposing an underlying layer. A first underlying layer is then dissolves or disintegrates to release fluoride compound 103 and to expose a subsequent underlying layer, and so forth, until all layers of separator 100 have been degraded within a predetermined time period.

In use, orthodontic separator 100 is inserted into a space between two adjacent teeth, as shown in FIG. 1A, thereby reducing the risk of dislodgement of orthodontic separator 100 with no significant irritation of the mouth.

Orthodontic separator 100 may be inserted manually, or using dental floss, or with the use of a suitable gripping/dental device, such as placement forceps, band placement pliers or tweezers. Insertion of orthodontic separator 100 may be performed by the subject or by any caregiver or auxiliary caregiver in the medical health care field, such as a doctor, dentist, nurse or parent of a subject.

Separator 100 may include a polymer wherein as a result of dissolution or disintegration, hydrolysis or absorption, removal of orthodontic separator 100 is not required. In embodiments wherein substrate 101 includes a non-absorbable polymer, removal of orthodontic separator 100 is required and may be performed by the subject or by any caregiver or auxiliary caregiver.

In some embodiments, substrate 101 further includes a second polymer in addition to PVOH or a different type of PVOH such as one having a different degree of hydrolyzation. Non-limiting examples of polymers which are suitable as the second polymer include acrylates, methacrylates, acrylic acids, methacrylic acids, alkyl polymers and combinations thereof.

In some embodiments, substrate 101 further includes additional excipients such as flavoring agents, coloring agents and the like.

FIG. 2 shows an alternative embodiment of the orthodontic separator 100 of the present invention, wherein ring 102 (FIGS. 1A and 1B) further includes or is in the form of a disk or plate 104. Plate 104 at least partially includes substrate 101, which may include a fluoride compound 103 incorporated therein. Plate 104 may be applied at any suitable intraoral location, such as between two adjacent teeth, as described above with reference to ring 102.

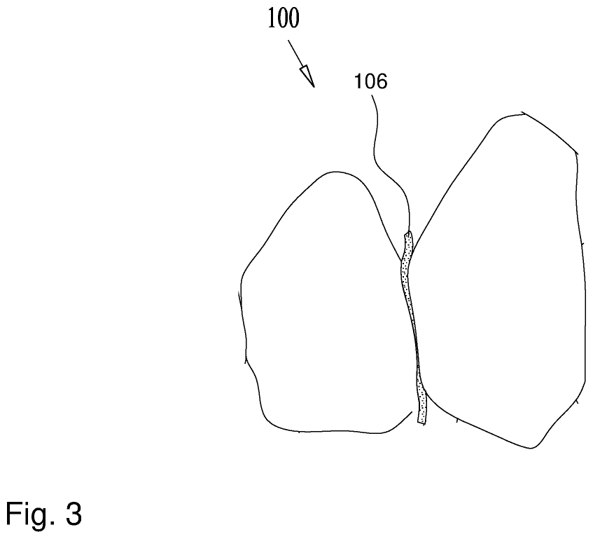

FIG. 3 shows an alternative embodiment of the orthodontic separator 100 of the present invention, wherein ring 102 includes or is in the form of a filament 106. In some embodiments, filament 106 includes substrate 101 having fluoride compound 103 incorporated therein. Filament 106 may be applied at any suitable intraoral location, such as between two adjacent teeth, as described above with reference to ring 102 or plate 104.

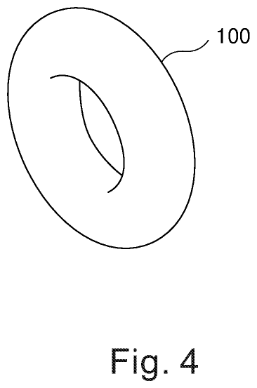

FIG. 4 shows orthodontic separator 100 in the form of ring 102 being a toroidal-shaped ring, which may be a convenient form keeping the separator secured between adjacent teeth.

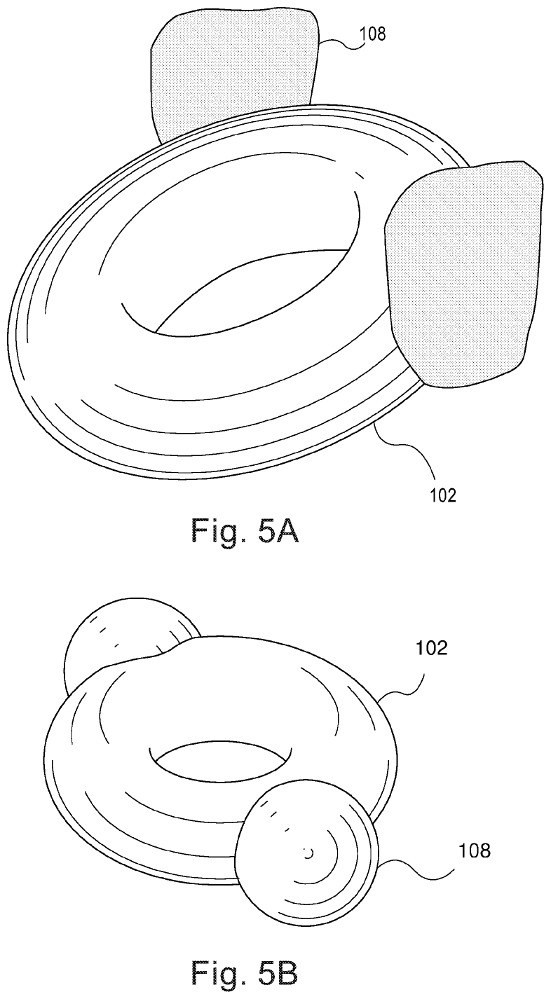

FIG. 5A shows orthodontic separator 100 as in FIG. 4, however wherein ring 102 includes one or more wings 108; two illustrated, and the wings can take a variety of shapes/sizes. Wings 108 may be useful in securing ring 102 in place, in particular to prevent the ring from spinning and/or to maintain the ring in a generally ring-like shape, and/or to ease installation of the ring and/or for helping visualize separator 100 while ring 102 portion thereof may be hidden between teeth. Additionally or alternatively, wings 108 can be useful to provide more volume (more material) for storing active ingredient 103.

FIG. 5B illustrates an exemplary shape of wings 108, exemplified by spheres. The spherical shape can provide more volume to store active ingredient 103, as well as potentially providing the potential benefits listed above.

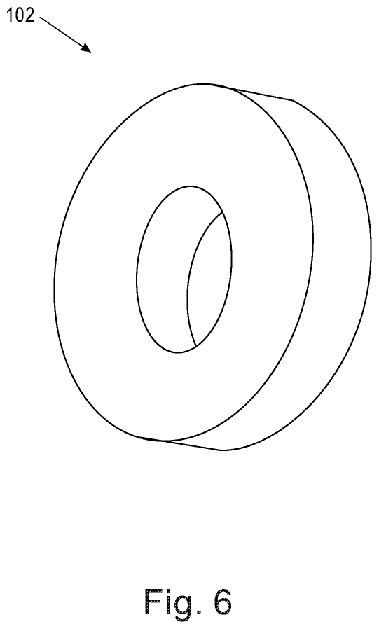

FIG. 6 shows orthodontic separator 100 having a 3-D washer-shape, which, in some embodiments, can be constituted by a sponge-like material.

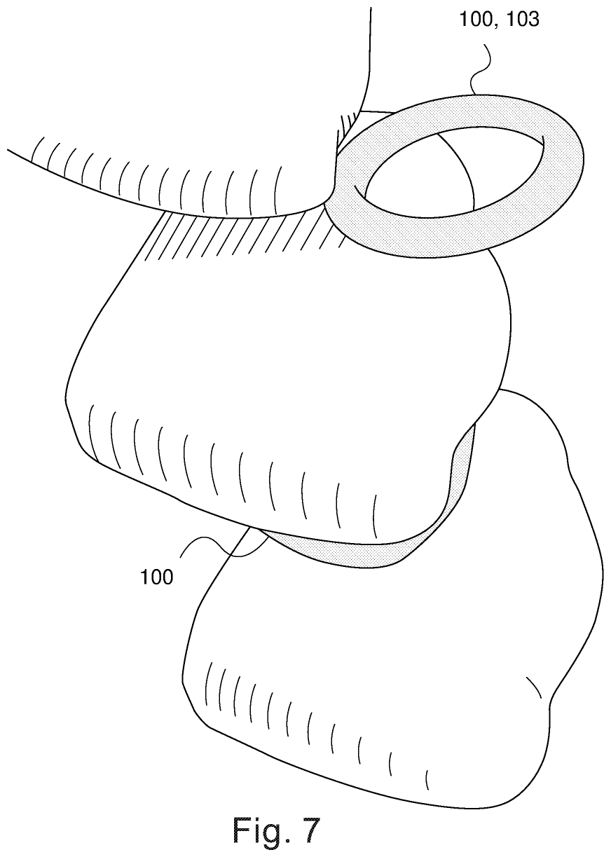

FIG. 7 shows an exemplary orthodontic separator of the present invention being inserted between teeth. The orthodontic separator is exemplified by ring 102, which may be a convenient form for fitting in and around gaps between adjacent teeth.

In any of the embodiments disclosed herein, substrate 101 may further includes, in addition to active ingredient 103, an additional active agent/excipient. The additional active agent may be configured to act locally at the intraoral location for treatment of a condition of the oral cavity or may be released into the saliva to be swallowed for systemic administration or absorbed through oral membranes.

Non-limiting examples of suitable additional active agents include anti-fungal agents, to treat fungal infection of the oral cavity; anesthetic agents (such as benzocaine or xylocaine); analgesic agents; agents for the treatment of mouth ulcers (aphthae); antibacterial agents; and anti-inflammatory agents.

In some embodiments, substrate 101 further includes silver ions to provide an antibacterial effect. In some such embodiments, the silver ions are provided as silver diamine fluoride (SDF, CAS nr. 34445-07-3). The silver ions may be present at very low concentrations, such that the silver color of the material is not readily visible.

A method for forming orthodontic separator 100 by dissolving a polymer in a solvent to form a polymer composition includes; forming a film from the polymer composition to provide substrate 101; and incorporating a fluoride compound or the like, for example sodium fluoride, into the substrate. The compound may be incorporated into substrate 101 by addition to the polymer composition prior to forming the film or may be incorporated into the substrate by adding to the formed film.

In some embodiments, the film is formed by casting, such as thermal casting.

In some embodiments, the fluoride compound includes a fluoride anion with a suitable cation whereby release of the fluoride anion can be achieved by ion exchange with one or more anions present in saliva. The ion exchange process may be facilitated by rinsing the oral cavity with an anion-containing mouthwash to increase the fluoride concentration.

During the course of treatment, any suitable number of orthodontic separators 100 may be used simultaneously and/or serially to achieve a desired effect.

Use of PVOH to form a substrate produced clear, hard films. Dissolution or disintegration of polymers is not only dependent on polarity, as in small molecules, but also on the degree of crystallinity. Although this does not affect the final thermodynamic result, it does have a tremendous effect of the kinetics (rate) of the dissolution or disintegration, and thus can improve the production efficiency.

Dissolution/disintegration experiments were performed on aqueous solutions at 0.4% salinity at 25-28° C. on a vertical solution-mixing roller. The rates of dissolution or disintegration for PVOH is shown by the percentage polymer remaining on days 1, 2, 4, 7, 10, 12 and 14 are presented in Table 1.

TABLE 1
Dissolution or disintegration of PVOH in saline solution

| | Day | | | | | | |
|---|---|---|---|---|---|---|---|
| | 1 | 2 | 4 | 7 | 10 | 12 | 14 |
| PVOH (% | 100 | 98 | 89 | 74 | 61 | 41 | 39 |
| remaining in | | | | | | | |
| solution) | | | | | | | |

The results shown in Table 1 indicate that the rate of dissolution or disintegration for films produced using PVOH is satisfactory for at least a 2 week period. Further, the dissolution or disintegration rate is adjustable by use of different grades of the polymer and combinations thereof.

PVOH is available in a wide variety of grades, is approved for use in food, drugs and cosmetics industries and can be cross-linked. X-linking can make the polymer an elastomer/elastic, however it will greatly reduce solubility.

The substrate/film may be cast from compositions including 95-99.5% hydrolyzed PVOH with one of sodium fluoride, sodium monofluorophosphate or stannous fluoride at different concentrations.

To confirm uniformity of release rate, for each film/substrate, UV absorption at 540 nm was measured, to determine how well the NaF dispersed in (PVOH) substrate 101. UV absorption results are presented in Table 2.

TABLE 2
UV absorption of PVOH substrates including fluoride compounds

| PVOH concentration | Fluoride compound and | UV absorption |
|---|---|---|
| (wt % in solution) | concentration | (540 nm) |
| 5 | NaF (0.25 wt %) | 0 |
| 5 | NaF (0.5 wt %) | 0.1 |
| 5 | NaF (1 wt %) | 0.2 |
| 10 | NaF (0.1 wt %) | 0 |
| 10 | NaF (0.25 wt %) | 0 |
| 10 | NaF (0.5 wt %) | 0.3 |
| 10 | NaF (1 wt %) | 0.7 |
| 10 | NAFP | 0.1 |
| 10 | NAFP | 0.4 |
| 10 | NAFP | 0.9 |
| 10 | SnF2 (0.1 wt %) | 0.9 |
| 10 | SnF2 (0.2 wt %) | 1.2 |
| 10 | SnF2 (0.5 wt %) | 1.4 |

The data in Table 2 indicate that films cast from PVOH with sodium fluoride (NaF) were found to provide the lowest UV absorption values as compared to those prepared using the other fluoride compounds tested, thereby indicating a tendency for good dispersion of the NaF in the PVOH.

The rate of fluoride release was tested using conductivity meters specifically tuned to fluoride content. Films including hydrolyzed PVOH were cast from a solution of PVOH with sodium fluoride. The films were dried in an oven at 110° C. A film having a significantly homogenous distribution of NaF was obtained.

Fluoride release rates using other grades (MW) of PVOH showed similar results.

Without limitation to theory, it is believed that a particularly significant factor in the release of fluoride is the formation of small capillaries (pores) in the film, during the production process allowing for the entry of water into the film body (substrate).

To increase/improve elasticity, the PVOH-SnF composition may include one or more plasticizers and/or softeners, as noted herein-above. Exemplary plasticizers include ethylene glycol; glycerol; and diethylene glycol.

The use of polymeric glycols (i.e. long chain glycols) may provide the same increase in plasticity as that obtained using ethylene glycol but may affect the dissolution or disintegration rate.

Various grades (MW) of polyethylene glycol (PEG) may provide the PVOH-composite films/substrates with an acceptable level of elasticity as that obtained using ethylene glycol, however, they may to somewhat shorten the dissolution or disintegration time of the PVOH.

As understood from the disclosure herein, the present orthodontic separator may include an ingredient or composition configured to release of an active ingredient, in particular fluoride compositions can be produced according to the principles of the present invention, and include a separator made from an aqueous solution.

Fluoride release rates were found to be very similar to the polymer dissolution/disintegration rates, thereby indicating a generally homogeneous composite.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

1-50. (canceled)

51. An orthodontic separator comprising: a substrate comprising at least one resilient material configured to produce and maintain an outward force for a predetermined time period between adjacent teeth, wherein the substrate comprises polymer fibers having a fluoride compound incorporated therein or that coats a surface thereof, wherein said substrate dissolves or disintegrates or degrades within said predetermined time period when disposed between those adjacent teeth.

52. The separator of claim 51, wherein the substrate comprises a plurality of layers including polymer fibers having a fluoride compound incorporated therein or that coats a surface thereof, wherein said plurality of layers are arranged concentrically and configured such that the plurality of layers of the substrate successively dissolve or disintegrate or degrade within said predetermined time period when disposed between those adjacent teeth.

53. The separator of claim 51, wherein the substrate comprises, singularly or in any combination: polyvinyl alcohol (PVOH); polyacrylic acid (PA A) and derivatives thereof, including polyacrylamide (PA Am) and polymethacrylamide (PM A A); polyethylene oxide (PEO); silicone elastomers; crosslinked hyalauronic acid (X-HYA); and blends of polyglycolic acid, polylactic acid, and caprolactone.

54. The separator of claim 51, wherein the substrate comprises a cast film or lamination prepared from a solution including water as a solvent.

55. The separator of claim 51, wherein the substrate comprises at least one resilience or flexibility enhancer.

56. The separator of claim 54, wherein the at least one enhancer comprises a plasticizer or softening agent.

57. The separator of claim 51, wherein the substrate includes a dental care agent;
and/or therapeutic agent; and/or active ingredient.

58. The separator of claim 51, wherein the substrate comprises a coating.

59. The separator of claim 58, wherein the coating is a lubricating coating.

60. The separator of claim 51, wherein the substrate comprises an anti-microbial coating.

61. The separator of claim 51, wherein said predetermined time period is at least one week.

62. A method of forming an orthodontic separator, the method comprising: preparing a substrate comprising a resilient material configured to produce and maintain an outward force for a predetermined time period between adjacent teeth, forming the substrate from polymer fibers having a fluoride compound incorporated therein or that coats a surface thereof, wherein said substrate dissolves or disintegrates or degrades within said predetermined time period when disposed between those adjacent teeth.

63. The method of claim 62 further comprising forming the substrate from a plurality of layers including polymer fibers having a fluoride compound incorporated therein or that coats a surface thereof, wherein said plurality of layers are arranged concentrically, and configured such that the plurality of layers of the substrate successively dissolve or disintegrate or degrade within said predetermined time period when disposed between those adjacent teeth.

64. The method of claim 62, wherein preparing the substrate comprises, singularly or in any combination, incorporating polyvinyl alcohol (PVOH); polyacrylic acid (PAA) and derivatives thereof, including polyacrylamide (PA Am) and polymethacrylamide (PM A A);
polyethylene oxide (PEO); silicone elastomers; crosslinked hyaluronic acid (X-HYA); and
blends of polyglycolic acid, polylactic acid, and caprolactone to form the substrate.

65. The method of claim 62, wherein preparing the substrate comprises casting and/or laminating and/or extruding and/or injection molding and/or liquid injection molding and/or compression molding and/or reaction injection molding and/or pressing and/or drawing and/or die cutting.

66. The method of claim 62, wherein preparing the substrate comprises adding at least one resilience or flexibility enhancer.

67. The method of claim 62, wherein preparing the substrate comprises adding at least one plasticizer or softening agent.

68. The method of claim 62, wherein preparing the substrate comprises adding a dental care agent; and/or a therapeutic agent; and/or an active ingredient.

69. The method of claim 62 wherein preparing the substrate comprises applying a coating.

70. The method of claim 62, wherein preparing the substrate comprises compounding and extruding.
